(19) United States
(12) Patent Application Publication
(10) Pub. No.: US 2024/0390366 A1
(43) Pub. Date: Nov. 28, 2024

(54) FORMULATION AND METHOD FOR TOPICAL TREATMENT OF MYCOBACTERIUM ULCERANS IN BURULI ULCERS

(71) Applicant: MannKind Corporation, Danbury, CT (US)

(72) Inventors: Thomas Hofmann, Doylestown, PA (US); Brandon Banaschewski, Wayne, PA (US)

(21) Appl. No.: 18/714,456

(22) PCT Filed: Nov. 29, 2022
(86) PCT No.: PCT/US2022/080537

§ 371 Date: May 29, 2024

Related U.S. Application Data
(60) Provisional application No. 63/284,426, filed on Nov. 30, 2021.

(51) Int. Cl. A61K 31/498; A61K 9/00; A61K 9/107; A61K 47/12; A61K 47/24; A61K 47/26

(52) U.S. Cl. CPC A61K 31/498; A61K 9/0014; A61K 9/107; A61K 47/12; A61K 47/24; A61K 47/26

(57) ABSTRACT

Disclosed herein are pharmaceutical compositions and methods for the treatment and prophylaxis of bacterial infections of the skin, including, topical ulcers caused by Mycobacterium ulcerans in Buruli ulcers, and/or other topical infections and types of inflammation. In particular, the compositions comprise clofazimine, derivatives thereof, polymorphs thereof, and/or analogs thereof in a solution, suspension, creams, oils, emulsions, and ointments for topical application. Methods for making the compositions are also disclosed.

### TECHNICAL FIELD

Disclosed herein are compositions, process for making the compositions and methods for the treatment and prophylaxis of bacterial infections of the skin, including, topical ulcers caused by Mycobacterium ulcerans. In particular, the compositions comprise clofazimine in a solution, suspension, and ointments for topical application in topical infections, inflammation, and other skin conditions.

### BACKGROUND

Buruli ulcer is a severe skin disease caused by infection of the skin by Mycobacterium ulcerans, and is most commonly found across West and Central Africa. Buruli ulcer is a global orphan disease, with approximately 61,119 cases diagnosed globally between 2002-2017 (Yotsu et al., 2018). The disease is third most common mycobacterial disease after tuberculosis and leprosy, and is usually diagnosed in its later stages, when it has caused substantial damage and disability. Surgery is the primary treatment of choice.

Current World Health Organization (WHO) treatment recommendations include 8-week therapy with a combination of oral antibiotics, including, rifampicin and intramuscular injection of streptomycin. While this regimen is microbiologically effective, patients require hospitalization due to dosing frequency, and route of administration (Demange, et al. 2009). New oral-only therapeutic regimens are under investigation, but there have been no changes to WHO guidelines to date (reviewed by Yotsu, et al. 2018).

Clofazimine is classified as Biopharmaceutical Classification System (BCS) class 2 which is practically insoluble in water and shows high membrane permeability. Serious adverse effects are dose related, primarily affecting the gastrointestinal tract. Reddish-brown discoloration of the skin and conjunctiva of the eyes are common over the course of oral therapy, and gradually reversible on cessation of therapy. Oral clofazimine can also induce gastroenteritis. Preclinical models have demonstrated a benefit of treating Mycobacterium ulcerans infected mice with a combination of oral rifampicin and clofazimine (Converse et al. 2015, Converse et al. 2018). Combination treatment of skin disease with oral antibiotics can cause severe side effects, and no current treatment with antibiotics has proven to be effective for all forms of Mycobacterium ulcerans infection. The side effects amplify the imperative need and benefit to develop new treatments for dermal therapy.

### SUMMARY

Disclosed herein are methods and compositions for the treatment of skin infections comprising antibiotics, including, clofazimine to be delivered via an ointment, cream, solution or suspension spray for topical administration. The method comprises applying a topical dose of a composition to a patient having ulcers on his or her skin caused by a bacterial infection, including, Mycobacterium ulcerans. The method is advantageous as it facilitates patient treatment with a dose that is less toxic than treatment with oral tablets. In an example embodiment, a composition comprising clofazimine, a clofazimine polymorph, a clofazimine derivative or a clofazimine salt thereof, or combinations thereof, is applied directly to ulcerated tissue of the patient's skin, at the site of ulceration, to bring about therapeutic effect more quickly, with less toxic side effects and using lesser amounts of active agent than orally administered compositions.

In one embodiment, the method comprises administering to a patient in need of treatment a therapeutically effective dose of a clofazimine composition to be topically applied on the patient's skin. The clofazimine composition can be provided to the patient in the form of a neat drug, or a pharmaceutically acceptable derivative, polymorphs of clofazimine, or salt thereof. In some embodiments, the clofazimine composition comprises a pharmaceutically acceptable carrier or excipient. In certain embodiments, the clofazimine composition can comprise a solution; a suspension, which can be sprayed onto the ulcer; or as a topical ointment, foam, or cream, which can be applied directly to the ulcerated tissue or applied to an adhesive tape or wrap.

In some embodiments, a method of treatment is disclosed, comprising, administering to a subject in need a therapeutic amount of a composition comprising clofazimine and a pharmaceutically acceptable carrier and/or excipient, wherein the bacterial infection is of the Buruli type or other mycobacterial infection.

In a particular embodiment, the method comprises, applying to a subject diagnosed with positive Mycobacterium ulcerans infection, a therapeutically effective amount of a topical clofazimine composition comprising, clofazimine, a pharmaceutically acceptable derivative, polymorphs of clofazimine, or a salt of clofazimine, including, the hydrochloride salt of clofazimine, including, clofazimine acetate, clofazimine citrate, clofazimine phosphate, clofazimine oxalate, clofazimine sulfate, or combinations thereof, and a pharmaceutically acceptable excipient and/or carrier, to inhibit bacterial replication on an ulcer of the skin of a patient.

In one embodiment, the method of treatment comprises, administering to a subject a therapeutically effective amount of a topical clofazimine composition, wherein the clofazimine, a pharmaceutically acceptable derivative, salt thereof, or combination thereof, is in an amount of about 1 mg to about 30 mg; from about 1 mg to about 20 mg; from about 1 mg to about 10 mg; from about 3 mg to about 8 mg, or from about 2 mg to about 6 mg of clofazimine; derivative or salt thereof per dose in the composition to be delivered in a solution or a suspension daily for a predetermined time as required for healing of the ulcers. In an embodiment, the total amount of topical antibiotic can be applied one or more times a day as needed. The composition can be applied to cover the entire area of ulceration.

In one embodiment, the composition can include a pharmaceutically acceptable excipient and the clofazimine can comprise up to 50 mg per dosage to be administered to a subject in need of treatment, and delivered to the wound or ulcer by spraying the solution or suspension using a spraying or misting device, or by spreading the clofazimine ointment or cream in a dispensing tube or jar and spreading with a disposable spatula or device. In some embodiments, the topical clofazimine composition can be formulated into solution, suspension, lotion, paste, ointment, cream, gel, oil, band, aerosol, spray, foam, powder, and/or occlusive dressing.

In one particular embodiment, the solution or suspension can comprise a saline solution or an alcohol-based solution. In some embodiments, a viscous excipient is used in order to facilitate the application and retention of the clofazimine, clofazimine derivative, clofazimine salt or combinations thereof in the ulcerated region of the skin.

### DETAILED DESCRIPTION

In embodiments disclosed herein there is provided a method for the treatment of mycobacterium infections comprising the use of clofazimine solution, clofazimine suspension for applying to the ulcer dropwise, spraying directly as an aerosol, or by directly spreading clofazimine cream and clofazimine ointment for direct topical application to an ulcerated area on the skin of a subject. In particular, the ulcers on the skin are caused by Mycobacterium ulcerans, for example, in Buruli ulcers disease.

In an exemplary embodiment, the method comprises applying a topical clofazimine medication for the treatment of Buruli ulcers caused by Mycobacterium ulcerans comprising an antibiotic which is a lipophilic compound, fat soluble, having the general formula:

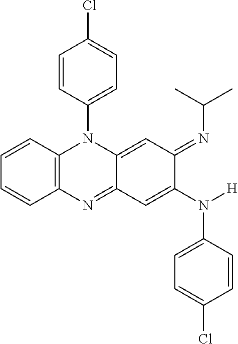

and one or more pharmaceutical acceptable excipients and/or carriers.

In one embodiment, the medication or formulation comprises clofazimine, a derivative thereof, a salt thereof, and/or combinations thereof, provided as micronized suspensions, including, surfactants, milled particles, and in which the milled drug particles are smaller than the aerosol droplets. In one embodiment, the topical medication is provided in the form of a liposomal formulation, including, lipophilic agents such as phospholipids, including, 1,2-distearoyl-sn-glycero-3-phosphocholine (DSPC) and/or dipalmitoylphosphatidylcholine (DPPC) or other existing liposomes. In some embodiments, the formulation can contain one or more ingredients including solvents, for example, ethanol or propylene glycol, dextran and cyclodextrin, and surfactants, including, polysorbate 80.

In one embodiment, a topical clofazimine formulation is provided for treating a Mycobacterium ulcerans infection comprising an ultra-pure Polysorbate 80 and/or one or more pharmaceutically acceptable excipients or carriers. In this embodiment, the clofazimine formulation provides a low allergic reaction; it has low toxicity and has low levels of peroxide compounds.

In some embodiments, the method comprises a composition for solid dispersion of the clofazimine, clofazimine salt, clofazimine derivative or clofazimine polymorphic compound. In one embodiment, the term solid dispersion refers to a group of solid products consisting of at least two different components, one generally a hydrophilic matrix and a hydrophobic drug compound. In an embodiment, the solid dispersion comprises a clofazimine compound, derivative or salt thereof and an acidic bioerodible polymer excipient, for example, a PVM/MA (poly (vinyl methyl ether-maleic anhydride)) copolymer. In this embodiment, the composition has superior dissolution and local or systemic availability. PVM/MA is a compound approved by the FDA for use in toothpastes and as denture adhesives and can provide topical adhesion of the formulation for best results in delivering the active compound.

In another embodiment, the method comprises administering a self micro-emulsifying drug delivery system (SMEDDS). In this embodiment, the method comprises providing to a patient a composition comprising a mixture of an oil, a surfactant and optionally, a co-solvent or co-surfactant that spontaneously forms a stable micro-emulsion upon dilution with water. The micro-emulsion can then be applied to the ulcerated skin by way of dropwise and spreading on the areas infected with the mycobacterium.

In specific embodiments, a dermal formulation for the treatment with topical clofazimine antibiotic can comprise one or more of the following ingredients or delivery systems, including, a surfactant such as polysorbate 80; an emulsifier such as oleic acid; a micro-milling drug compound; liposomes of 1,2-distearoyl-sn-glycero-3-phosphocholine (DSPC) and/or dipalmitoylphosphatidylcholine (DPPC), and/or cholesterol; poly (DL-lactide-co-glycolide) nanospheres; aerosol propellants; cellulose derivatives; and suspension of micronized drug and inactive ingredients.

In one embodiment, a method for treatment of dermal infections with Mycobacterium ulcerans, comprising, administering to a patient with said dermal infection, including, a skin ulcer a topical pharmaceutical composition comprising clofazimine, a clofazimine polymorph, a clofazimine derivative, a clofazimine salt, analogues thereof, and/or combinations thereof; and one or more pharmaceutically acceptable carriers or excipients.

In one embodiment, the topical pharmaceutical composition comprises a solution, suspension, lotion, paste, ointment, cream, gel, oil, band, aerosol, spray, powder, and/or occlusive dressing.

In another embodiment, the method of treatment comprises, providing a topical pharmaceutical formulation to a patient comprising a therapeutically effective dose of a clofazimine, a clofazimine derivative, a clofazimine salt and/or analogues in a suspension for spraying in an ulcerated area of the skin.

In an embodiment, the method of treatment comprising the pharmaceutical formulation for use is formed by emulsifying one or more clofazimine derivative, a clofazimine salt and/or analogues thereof into a suspension, with one or more excipients necessary for drug solubility in the topical formulation, and mixing a second solubility enhancing substance, including, a nonionic surfactant. In this embodiment, the formulation is formed by adding a secondary solubility enhancing substance, for example, a phospholipid, or a mixture of natural phospholipids, and wherein the topical pharmaceutical formulation comprises clofazimine, a clofazimine derivative, a clofazimine salt and/or analogues in a liposomal solubilized form.

In an alternate embodiment, the topical pharmaceutical composition comprises a content of clofazimine, a clofazimine derivative, a clofazimine salt and/or analogues of between 0.01 to about 100 mg per dose to be administered. In the embodiment wherein the topical pharmaceutical composition is an emulsion, the content of the emulsifying agent is <50% w/w, and >20% w/w water.

In one embodiment, the topical pharmaceutical composition wherein the content of a suspension is either an aqueous or alcoholic vehicle with solid particulate active content of clofazimine, a clofazimine derivative, a clofazimine salt and/or analogues as pharmaceutical ingredient(s) in the form of a gel, the gel contains any compositional mixture of water, acetone, alcohol, propylene glycol, and/or cellulose derivative.

In another embodiment, wherein the topical pharmaceutical composition is in a form of a foam, the content of the foam, aerosol, and/or spray contain any compositional mixture of a hydrocarbon propellants, nonpolar hydrocarbons, ethanol, acetone, hexadecyl alcohol, glycol ethers, polyvinylpyrrolidone and/or polyglycols.

A composition for treating a bacterial infection of the skin, for example, a Buruli ulcer is provided, wherein the composition contains emulsions or suspensions having uniform droplets with an average diameter of most about 1 μm and/or a polydispersity index of at most about 1 D.S. in some embodiments, the therapeutic composition is sterile and is free of solid particles comprising the active agent having a particle diameter of greater than or equal to 1 μm.

In one embodiment, the composition is a suspension of clofazimine, using micro milling of drug, and comprising polysorbate 80, 1,2-distearoyl-sn-glycero-3-phosphocholine (DSPC) and/or dipalmitoylphosphatidylcholine (DPPC) and/or cholesterol, and citric acid monohydrate, disodium edetate, sodium chloride, tri-sodium citrate dihydrate, and water.

In another embodiment, the composition is a suspension of clofazimine, including, hypertonic saline 3-7%, sodium bicarbonate, bismuth, gallium or d-amino-acids, which is for applying in dropwise manner or by spray from a device.

In another embodiment, the method comprises a composition having an antibiotic clofazimine concentration of about 1 mg/mL to about 3 mg/mL. In certain embodiments herewith, the composition is prepared having a pH of 3-10. In an embodiment, the composition can contain an inert buffer. In some embodiments, the topical composition can have an osmolality range of 200-700 mOsm/kg, and the ion concentration range of 31 to 300 mM.

In one embodiment, the composition can be topically administered to the skin of patients suffering from Mycobacterium ulcerans dermal infections, nontuberculous mycobacterial dermal infections, or other bacterial skin infections. In one embodiment, the composition can be applied topically to a body surface affected by bacterial infections in which the pathogen is susceptible to the respective antibiotic in the formulation.

In another embodiment, the composition comprising clofazimine, a clofazimine derivative, a clofazimine polymorph or clofazimine salt and/or clofazimine analogues as pharmaceutical ingredient(s) is provided for use in a diverse manner depending on the skin or mucous area to be treated and can be prepared as a medicament for oral, nasal, ophthalmic, pulmonary, parenteral, topical or mucosal application.

In one embodiment, a process for making an emulsion comprising clofazimine for topical use is provided, wherein the process provides an emulsion wherein the resultant emulsion comprises a percentage of emulsion droplets of <5 Pm of between 50% and 98%, or from 60-90%, and the emulsion droplets have a geometric standard deviation <2.2, or <1.8. In one embodiment, a process for making an emulsion yields emulsion droplets, wherein the percentage of emulsion droplets of <3.5 Pm is between 40% and 95%, or between 50-85%.

A method for making a stable pharmaceutical formulation for topical administration is provided comprising: emulsifying one or more of a clofazimine compound, a clofazimine derivative, a clofazimine salt and/or analogues thereof into a suspension with one or more excipients necessary for drug solubility to form a topical formulation, and mixing a secondary solubility enhancing substance including, a nonionic surfactant. In some embodiments, the second solubility enhancing substance is a phospholipid, or a mixture of natural phospholipids, and wherein the topical pharmaceutical formulation comprises the clofazimine, a clofazimine derivative, a clofazimine salt and/or analogues in a liposomal, solubilized form.

In certain embodiments, the method of making the stable pharmaceutical formulation for topical administration, further comprises a suspension of emulsion droplets of <5 Pm that are present in the suspension in a percentage of between 50% and 98% and more preferably 60-90% and the emulsion droplets have a geometric standard deviation <2.2 and preferably <1.8.

In another embodiment, the method of making the stable pharmaceutical formulation for topical administration, further comprises a suspension of emulsion droplets of <3.5 Pm that are present in the suspension in a percentage of between 40% and 95% and more preferably between 50-85% of the total droplets present.

In an alternate embodiment, a topical suspension as described above is provided which can be applied to various body surfaces, as anti-mycobacterial therapy for other types of infections other than ulcerated tissue. In some embodiments, a method of treating a mycobacterial infection is provided, which comprises applying a topical anti-mycobacterial pharmaceutical composition to a subject having a mycobacterial infection of the skin. In one embodiment, the anti-mycobacterial composition can comprise a suspension or solution comprising clofazimine, a clofazimine derivative, a clofazimine polymorph, a clofazimine analogue or combinations thereof, and one or more pharmaceutically acceptable excipients.

In yet an alternate embodiment, a topical suspension as described above is provided which can be applied to various body surfaces of a subject in need, as an anti-inflammatory therapy for other types of disease conditions of the skin. In one embodiment, a method of treating an inflammation of the skin is provided, which comprises applying a topical pharmaceutical composition to a subject having an inflammation of the skin which may be resultant from a mycobacterial infection or other pathogen of the skin. In one embodiment, the pharmaceutical composition can comprise a suspension or solution comprising clofazimine, a clofazimine derivative, a clofazimine polymorph, a clofazimine analogue or combinations thereof and one or more pharmaceutically acceptable excipients.

In one embodiment, a clofazimine solution or suspension can comprise from about 0.1 mg/mL to about 100 mg/mL. In some embodiments, a solution, suspension or foam comprising clofazimine, a clofazimine derivative, a clofazimine polymorph, a clofazimine analogue or combinations thereof is in a concentration of about 0.2 mg/L to about 10 mg/L; and one or more pharmaceutically acceptable carriers and/or excipients.

In an alternate embodiment, a method is provided for treating skin disease caused by a mycobacterium infection comprising applying to the infected skin region a topical pharmaceutical composition comprising clofazimine, a clofazimine derivative, a clofazimine polymorph, a clofazimine analogue or combinations thereof, in a combination therapy with one or more antibacterial agents, or antibiotics, wherein the antibacterial agent can be selected from rifampicin, clarithromycin, bacitracin, ciproflaxin, moxifloxacin, ethambutol, amikacin, azithromycin and levofloxacin. In one embodiment, the one or more antibacterial agents can be formulated together with the clofazimine compound, or applied separately to the infected area in its own formulation and at the same time of application of the clofazimine or at different intervals during treatment, or by a different method including oral tablets or capsules, or by intravenous administration. The combination therapy may facilitate treatment of the disease.

### Example 1

Development of a clofazimine liposomal formulation is as follows: Uni-lamellar liposomes with mean average diameter of 100 nm and polydispersity of 0.4-0.5 are formed by the addition of an emulsifier/solubilizer such as Polysorbate 80 (HX2)™ and/or an alternate tenside. Polysorbate 80 (HX2)™ to clofazimine compound in a suspension, including 0.9% NaCl solution or suspension. Polysorbate 80 (HX2)™ gives the best result for topical formulation, for example, low allergic reaction, low toxicity and low level of peroxide compounds. The clofazimine in solid form can be first dissolved in 75% acetic acid prior to emulsification with polysorbate 80.

The polysorbate 80 is frequently used in pharmaceutical formulation as an emulsifier, solubilizer and stabilizer and can be added to the formulation at room temperature. The formulations are stable at room temperature, and at extreme tropical temperatures of ˜40° C. and can be used to apply to target dosing frequency, for example, for daily topical application until the ulcer or wound area is healed. The daily concentration of antibiotic in the formulation is dependent on the severity of the ulcers, for example, a suspension can comprise about 0.1-1.0 mg/g.

Viscosity of the formulation will be dependent upon final topical formulation. For example, the viscosity of aerosol sprays will differ from the viscosity of gels. The formulation is stable for 6+ months at temperatures between −20° C. and 60° C.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A topical pharmaceutical composition for treatment of dermal infections with Mycobacterium ulcerans, comprising clofazimine, a clofazimine polymorph, a clofazimine derivative, a clofazimine salt and/or analogues thereof; and one or more pharmaceutically acceptable carriers or excipients.

2. The topical pharmaceutical composition of claim 1, wherein the topical pharmaceutical composition comprises a solution, suspension, lotion, paste, ointment, cream, gel, oil, band, aerosol, spray, powder, and/or occlusive dressing.

3. The topical pharmaceutical composition of claim 1, further comprising a therapeutically effective dose of a clofazimine, a clofazimine polymorph, a clofazimine derivative, a clofazimine salt and/or analogues thereof in a suspension for dripping or spraying on an ulcerated area of the skin.

4. The topical pharmaceutical composition of claim 2, wherein the content of clofazimine, a clofazimine polymorph, a clofazimine derivative, a clofazimine salt and/or analogues is between 0.01 to about 100 mg per dose.

5. The topical pharmaceutical composition according to any one of the preceding claims, wherein the content of the emulsifying agent is less than 50% w/w, and greater than 20% w/w water.

6. The topical pharmaceutical composition according to any one of the preceding claims, wherein the content of a suspension is either an aqueous or alcoholic vehicle with solid particulate active content of clofazimine, a clofazimine polymorph, a clofazimine derivative, a clofazimine salt and/or analogues as pharmaceutical ingredient(s).

7. The topical pharmaceutical composition according to any one of the preceding claims, wherein the content of the gel contains any compositional mixture of water, acetone, alcohol, propylene glycol, and/or cellulose derivative.

8. The topical pharmaceutical composition according to any one of the preceding claims, wherein the content of the foam, aerosol, and/or spray contain any compositional mixture of the composition according to claims 2-7, with the addition of hydrocarbon propellants, nonpolar hydrocarbons, ethanol, acetone, hexadecyl alcohol, glycol ethers, and/or polyglycols.

9. The topical pharmaceutical composition according to any one of the preceding claims, wherein the composition contains emulsions or suspensions with an average diameter of most about 1 μm and/or a polydispersity index of at most about 1 D.S.

10. The topical pharmaceutical composition according to any one of the preceding claims, wherein the composition is sterile and is free of solid particles of active agent having a particle diameter of greater than or equal to 1 μm.

11. The topical pharmaceutical composition according to any one of the preceding claims, wherein the composition is a suspension of clofazimine, using micro milling of drug, and the use of Polysorbate 80, dipalmitoylphosphatidylcholine (DPPC), and/or 1,2-diestearoyl-sn-glycero-3-phosphochyoline (DSPC) and/or cholesterol, and citric acid monohydrate disodium edetate, sodium chloride, tri-sodium dehydrate, and water for application.

12. The topical pharmaceutical composition according to any one of the preceding claims, wherein the composition is a suspension of clofazimine, including hypertonic saline 3-7%, sodium bicarbonate, bismuth, gallium or d-amino-acids.

13. The topical pharmaceutical composition according to any one of the preceding claims, wherein the composition has an antibiotic concentration of 1 mg/mL.

14. The topical pharmaceutical composition according to any one of the preceding claims, wherein the composition has a pH of 3-10.

15. The topical pharmaceutical composition according to any one of the preceding claims, wherein the composition contains an inert buffer.

16. The topical pharmaceutical composition according to any one of the preceding claims, wherein the composition has an osmolality range of 200-700 mOsm/kg, and the ion concentration range of 31 to 300 mM.

17. The topical pharmaceutical composition according to any one of the preceding claims, wherein the composition can be topically administered to the skin of patients suffering from Mycobacterium ulcerans dermal infections, nontuberculous mycobacterial dermal infections, or other bacterial skin infections.

18. The topical pharmaceutical composition according to any one of the preceding claims, wherein the composition can be applied topically to any body surface affected by bacterial infections in which the pathogen is susceptible to the respective antibiotic in the formulation.

19. Use of a composition, according to any one of claims above, prepared as a medicament for oral, nasal, ophthalmic, pulmonary, parenteral, topical, or mucosal application.

20. A method for making a stable pharmaceutical formulation comprising: emulsifying one or more of a clofazimine compound, a clofazimine derivative, a polymorph, a clofazimine salt and/or analogues thereof into a suspension with one or more excipients necessary for drug solubility to form a topical formulation, and mixing a second solubility enhancing substance including, including a nonionic surfactant to form emulsion droplets.

21. The method of claim 20, wherein the second solubility enhancing substance is a phospholipid, or a mixture of natural phospholipids, and wherein the topical pharmaceutical formulation comprises the clofazimine, a clofazimine derivative, a clofazimine salt and/or analogues in a liposomal solubilized form.

22. The method of 20, wherein the stable pharmaceutical formulation further comprises emulsion droplets of <5 Pm are present in the suspension in a percentage of between 50% and 98% and more preferably 60-90% and the emulsion droplets have a geometric standard deviation <2.2 and preferably <1.8.

23. The method of claim 21, wherein the emulsion droplets of <3.5 Pm are present in the suspension in a percentage of between 40% and 95% and more preferably between 50-85%.
